(19) United States
(12) Patent Application Publication
(10) Pub. No.: US 2022/0273298 A1
(43) Pub. Date: Sep. 1, 2022

(54) SELF-LOCKING SUTURE STRAND

(71) Applicants: Filippo Concetto Chillemi, Duluth, GA (US); Wesley Alan Kent, Atlanta, GA (US)

(72) Inventors: Filippo Concetto Chillemi, Duluth, GA (US); Wesley Alan Kent, Atlanta, GA (US)

(21) Appl. No.: 17/661,313

(22) Filed: Apr. 29, 2022

Related U.S. Application Data
(60) Provisional application No. 63/200,316, filed on Mar. 1, 2021.

(51) Int. Cl. A61B 17/06; A61B 17/04

(52) U.S. Cl. CPC A61B 17/06166; A61B 17/0401; A61B 2017/00579

(57) ABSTRACT

A novel suture strand is herein disclosed. The suture strand includes a suture body, a hole, and multiple barbs. The hole is formed transversely through a first portion of the suture body. The barbs are coupled to a second portion of the suture body and are designed to allow the second portion of the suture body to pass through the hole in a first direction and prevent movement of the second portion of the suture body in an opposite, second direction.

### CROSS-REFERENCE TO RELATED APPLICATION

This application claims the benefit of priority of U.S. provisional application No. 63/200,316, filed Mar. 1, 2021, the contents of which are herein incorporated by reference.

### BACKGROUND OF THE INVENTION

The present invention relates to suture strands and, more particularly, to a self-locking surgical suture strand, utilizing barbs passing through a hole, eliminating the need for knot-tying.

Surgical suture knot-tying is challenging, especially arthroscopically, and the knot is often the point of failure, risking the overall surgical outcome. Before the present invention, no surgical sutures allowed the suture to self-lock, thus eliminating the need for knot-tying and gaining appropriate fixation.

Other suture products require knot-tying by the surgeon, which is often the point of failure as the knot comes apart with increased force. Other implants self-lock sutures by pressing the suture against soft tissue or bone, which can loosen with time and fail. Knot-tying failure due to increased forces or suture loosening due to failed fixation continues to be a point of failure in the surgical world. Other solutions, such as loops with protrusions that engage with the loops, are also suboptimal because they do not provide sufficient locking strength and require excessive material to form the suture.

As can be seen, there is a need for a suture strand that addresses the problems mentioned above.

### SUMMARY OF THE INVENTION

In one aspect of the present invention, a suture strand is disclosed that includes: a suture body; a hole formed transversely through a first portion of the suture body; and a plurality of barbs coupled to a second portion of the suture body, the plurality of barbs being configured to allow the second portion of the suture body to pass through the hole in a first direction and prevent movement of the second portion of the suture body in an opposite, second direction.

In certain embodiments, each barb defines a barb width and the opening defines an opening width, with the barb width being greater than the opening width.

In certain embodiments, the hole comprises elastin fibers.

In certain embodiments, the hole is deformable to permit the second portion of the suture body to pass through the hole in the first direction.

In certain embodiments, the hole is elastically returnable to an original hole size which, in conjunction with the plurality of barbs, prevents movement of the suture body in the second direction.

In certain embodiments, the hole comprises elastin fibers.

In certain embodiments, the plurality barbs are lined serially and define a plurality of sets of barbs.

In certain embodiments, the suture body comprises biocompatible polyethylene fibers combined with silicone or polyester.

In certain embodiments, the plurality of barbs are sewn onto the suture body.

In another aspect of the present invention, a method of securing a suture strand is disclosed, with the method including the steps of: providing the suture strand, the suture strand comprising: a suture body; a hole formed transversely through a first portion of the suture body; and a plurality of barbs coupled to a second portion of the suture body; passing the second portion of the suture body through the hole in a first direction, with the plurality of barbs preventing movement of the second portion of the suture body in an opposite, second direction.

These and other features, aspects and advantages of the present invention will become better understood with reference to the following drawings, description, and claims.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIG. 1 is a perspective view of a first embodiment of the present invention, showing an unsecured position;

FIG. 2 is another perspective view of the first embodiment of the present invention, showing a secured position;

FIG. 3 is a schematic view of a slight modified first embodiment 10A of the present invention, showing how a first end is looped through a second end to result in the secured position;

FIG. 4 is a schematic view of a second embodiment 20 of the present invention, showing an alternative design 21 to the barbs shown in FIGS. 1-3; and

FIG. 5 is a plan view of a third embodiment 30 of the present invention, showing an alternative design 31 to the barbs shown in FIGS. 1-3.

### DETAILED DESCRIPTION OF THE INVENTION

The subject disclosure is described with reference to the drawings, wherein like reference numerals are used to refer to like elements throughout. In the following description, for purposes of explanation, numerous specific details are set forth in order to provide a thorough understanding of the present disclosure such that one skilled in the art will be enabled to make and use the present invention. However, it may be evident that the present disclosure may be practiced without some of these specific details. For the purpose of clarity, technical material that is known in the technical fields related to the present invention has not been described in detail so that the present invention is not unnecessarily obscured.

Broadly, an embodiment of the present invention provides a suture strand that includes a suture body defining (1) an opening and (2) a plurality of barbs disposed on the suture body and configured to be inserted through the opening in a single direction.

The surgical suture strand of the present invention is self-locking and utilizes various-shaped barbs that pass through a hole at the end or within the length of the suture. Once these barbs pass through the hole in an antegrade fashion, the barb's shape and size will prohibit the suture from passing back through the hole in retrograde. This blockage in the retrograde passage will essentially self-lock the suture and eliminate the need for knot-tying. Before the present invention, there was no surgical grade suture strand on the market that exhibited the ability to self-lock by design, eliminating the need for knot-tying.

Advantageously, the present invention provides a self-locking suture based on barbs being passed through a hole, eliminating the need for knot-tying. The barb becomes fixated through the hole and cannot pass in a retrograde fashion. The present invention further includes a method of using and making this self-locking suture. This fixation point can withstand higher forces before failure than conventional knot-tying and doesn't require any implant assistance in fixation as it “locks” onto itself by design.

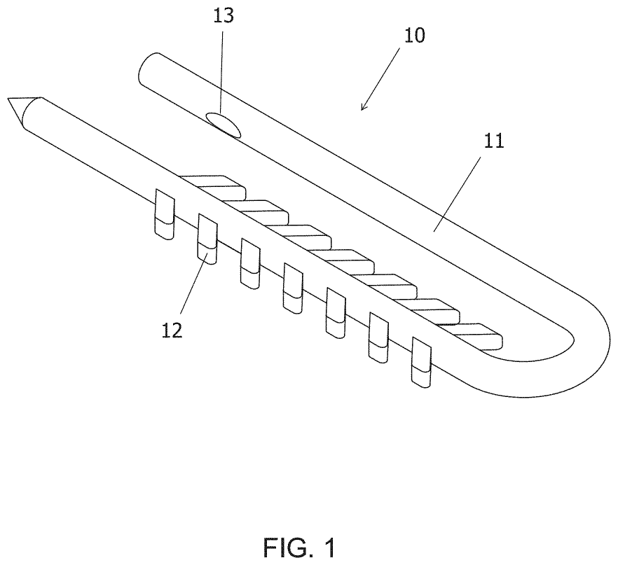

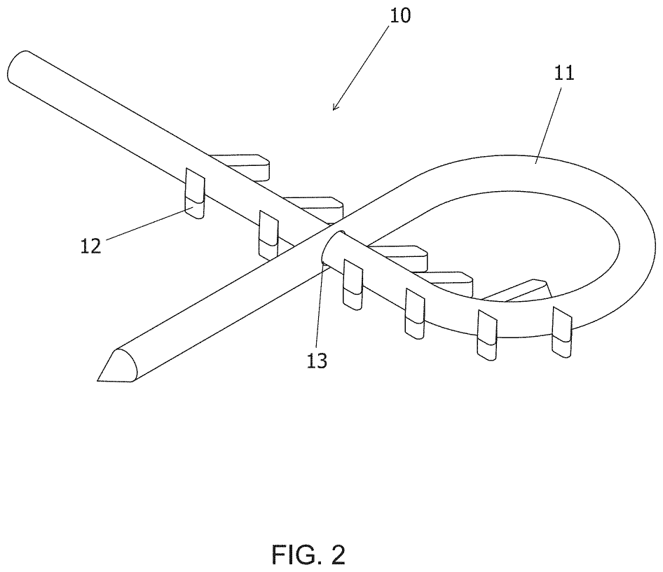

Referring now to FIGS. 1-5, a self-locking suture includes a suture body 11 with an end that includes a plurality of barbs 12 (selected from a plurality of shapes such as but not limited to: pronged, triangular, pear-shaped, bead-shaped, etc.) lined serially and designed to pass through a hole 13 (provided either at the end or along a length of the suture body 11) in an antegrade fashion. The serial line of barbs 12 is accomplished in such a way to define serially lined sets of barbs 12 (e.g., comprised of 2-4 barbs per set) along a portion of the suture body 11 in a length direction thereof. As seen in FIGS. 1 and 2, a width of each barb 12 or set of barbs 12 is greater than a width of the opening 13.

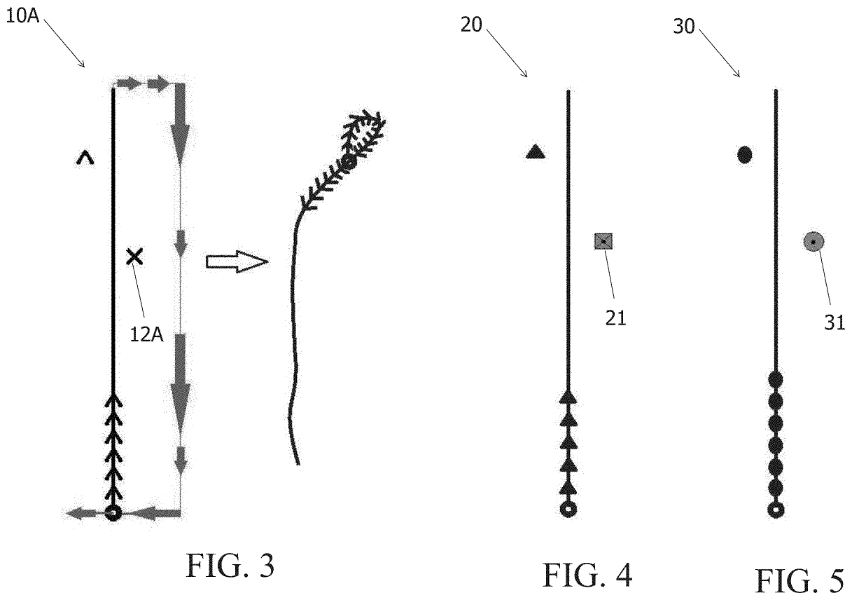

In certain embodiments, the suture strand 10 may be formed from biocompatible polyethylene fibers combined with silicone and/or polyester. The hole 13 may contain some elastin fibers. The elastin in the hole 13 will allow some deformation, allowing the larger barbs 12 to pass through. Once through, the hole 13 will return to its original size, locking the barbs 12 and preventing retrograde passage. As shown in FIGS. 3-5, the barbs 12 can be of various sizes and shapes, and the hole 13 can be of various sizes and diameters. The hole 13 can be defined at the end of the suture strand body 11 or within its length, depending on the need for suture 10 ends after fixation. As those with skill in the art will appreciate, the figures (e.g., FIGS. 3-5) do not necessarily depict exactly how the structure of the present invention will ornamentally appear, but rather is merely intended to supplement the present disclosure to provide the reader a better understanding of the present invention.

In use, fixation is created by passing one end of the suture 10 through the hole 13 and pulling it through until the barbs 12 start passing through. The barbs 12 will be able to pass through the hole 13 without difficulty. However, once through, due to the barb's shape, it will be unable to pass in a retrograde fashion back through the hole 13, as described above. This will prevent the suture 10 from loosening, therefore locking it without the need for knot-tying.

While methods of making and using the present invention would be readily apparent to those with skill in the art from the preceding disclosure, other methods may include the following. To make embodiments of the present invention, the design modifications described herein can be made in any appropriate fashion by modifying existing suture strand designs. These design modifications may, for example, be woven into current suture strands, creating the needed barbs 12. Further, a hole 13 may be formed in the suture body 11 beyond the barbs 12 to permit the ability of self-locking. To use embodiments of the present invention, the suture 10 will already be placed into an anchor, or the surgeon can place it into an anchor. The surgeon then must place the suture end (including the barbs 12) through the hole 13 and pull the barbs 12 through the hole 13 to cinch the suture 10 to the desired tightness.

While one or more preferred embodiments are disclosed, many other implementations will occur to one of ordinary skill in the art and are all within the scope of the invention. Each of the various embodiments described above may be combined with other described embodiments to provide multiple features. Furthermore, while the preceding describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

While apparatuses and methods are described in terms of “comprising,” “containing,” or “including” various components or steps, the apparatuses and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A suture strand comprising:
a suture body;
a hole formed transversely through a first portion of the suture body; and
a plurality of barbs coupled to a second portion of the suture body, the plurality of barbs being configured to allow the second portion of the suture body to pass through the hole in a first direction and prevent movement of the second portion of the suture body in an opposite, second direction.

2. The suture strand of claim 1, wherein each barb defines a barb width and the opening defines an opening width, with the barb width being greater than the opening width.

3. The suture strand of claim 2, wherein the hole comprises elastin fibers.

4. The suture strand of claim 2, wherein the hole is deformable to permit the second portion of the suture body to pass through the hole in the first direction.

5. The suture strand of claim 4, wherein the hole is elastically returnable to an original hole size which, in conjunction with the plurality of barbs, prevents movement of the suture body in the second direction.

6. The suture strand of claim 5, wherein the hole comprises elastin fibers.

7. The suture strand of claim 1, wherein the plurality barbs are lined serially and define a plurality of sets of barbs.

8. The suture strand of claim 7, wherein each set of barbs defines a barb width and the opening defines an opening width, with the barb width being greater than the opening width.

9. The suture strand of claim 8, wherein the hole is deformable to permit the second portion of the suture body to pass through the hole in the first direction.

10. The suture strand of claim 9, wherein the hole is elastically returnable to an original hole size which, in conjunction with the plurality of sets of barbs, prevents movement of the suture body in the second direction.

11. The suture strand of claim 10, wherein the hole comprises elastin fibers.

12. The suture strand of claim 1, wherein the suture body comprises biocompatible polyethylene fibers combined with silicone or polyester.

13. The suture strand of claim 1, wherein the plurality of barbs are sewn onto the suture body.

14. A method of securing a suture strand, the method comprising the steps of:
providing the suture strand, the suture strand comprising:
a suture body;
a hole formed transversely through a first portion of the suture body; and
a plurality of barbs coupled to a second portion of the suture body;
passing the second portion of the suture body through the hole in a first direction, with the plurality of barbs preventing movement of the second portion of the suture body in an opposite, second direction.

15. The method of claim 14, wherein, during the passing step, the hole deforms.
